(19) United States
(12) Patent Application Publication
(10) Pub. No.: US 2007/0191609 A1
(43) Pub. Date: Aug. 16, 2007

(54) PROCESS FOR PREPARATION OF CLOPIDOGREL BISULPHATE FORM-1

(75) Inventors: Venkat Reddy ALLA, Hyderabad (IN); Kameshwara Rao VYAKARANAM, Hyderabad (IN); Aruna Kumari SIRIGIRI, Hyderabad (IN); Srinivas Reddy BODAPATI, Hyderabad (IN); Ranadheer Reddy BILLA, Hyderabad (IN); Saikrishna Reddy GUDIBANDI, Hyderabad (IN); Raghumitra ALLA, Hyderabad (IN)

Correspondence Address: Ramesh Babu Vishwanath, VISHWANATH & COMPANY, Advocates & Attorneys,, # 48 SRT, 1st Floor, Prakash Nagar, Begumpet,, Hyderabad, omitted

(73) Assignee: LEE PHARMA LIMITED, Hyderabad (IN)

(21) Appl. No.: 11/307,663

(22) Filed: Feb. 16, 2006

(30) Foreign Application Priority Data
Feb. 13, 2006 (IN) 223/CHE/2006

(51) Int. Cl. C07D 498/02

(52) U.S. Cl. 546/114

(57) ABSTRACT

Disclosed herein is a cost effective and industrially feasible process for the preparation of (+) Clopidogrel bisulphate. The present invention further discloses a novel method of precipitation of (+) Clopidogrel bisulphate Form I directly from solvent mix of methanol and acetone in presence of sulfuric acid at a temperature of 25-40° C.

### FIELD OF THE INVENTION

The present invention relates to cost effective and industrially feasible process for the preparation of Clopidogrel bisulphate Form I.

### BACKGROUND AND PRIOR ART

Clopidogrel is the international non-proprietary name of (S)-(+)-Methyl (2-chlorophenyl)-(6,7-dihydro-4H-thieno[3,2-c]pyrid-5-yl)acetate which is having the following structure.

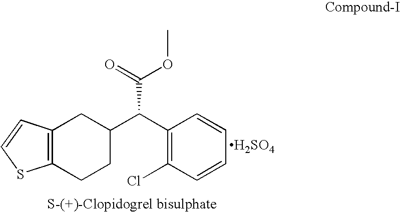

Recent studies have shown that Clopidogrel is more effective in blocking platelet aggregation than asprin and is much gentler on the gastrointestinal tract. Clopidogrel is more effective than asprin even at much lower dosage. Clopidogrel is administered as its bisulphate salt. The enantiomer (S)-(+) Clopidogrel is particularly preferred since it is the pharmaceutically active compound. Clopidogrel is administered as its bisulfate salt and currently being marketed as PLAVIX.™. Tablets.

Clopidogrel was first reported by Sanofi in EP99802 and was claimed as racemic product. European patent 281 459(herein after referred '459 patent), also filed by Sanofi, disclosed the enantiomers of tetrahydro thieno pyridine derivatives and its pharmaceutically acceptable salts. EP 281459 (corresponding to U.S. Pat. No. 4,847,265) described the separation of the optical isomers from the racemic clopidogrel base by using (+)-10-camphor sulphonic acid. The enantiomer (S) Clopidogrel is particularly preferred since it is therapeutically more active than its (−) isomer.

EP99802 discloses a method for preparation of Clopidogrel bisulfate. JP 4230387 discloses the preparation of (+) Clopidogrel bisulphate in detail with few improvements and modifications.

The polymorphic forms I and II for Clopidogrel bisulfate were first described in W099/6591 5. This patent application reported that Polymorphic Form-I was prepared according to the method described in EP 281459. The study of polymorphic forms I, II, III, IV, V and VI and its preparations are extensively disclosed in WO 03051362. In all the above mentioned patents and in all prior arts, Form-I was prepared by separation of isomer from clopidogrel base via camphor sulfonic acid route in the final stage.

From the teaching of the prior art it is amply clear that there remains scope to further develop process for preparation of (S)-Clopidogrel bisulphate, which is of high chiral purity and eliminate the need for further resolution. The lacuna and scope in the prior art lead us to carry out the present invention.

Hence, the present invention is aimed to provide a cost effective, simple and feasible industrial method to prepare (S)-Clopidogrel bisulphate Form I with good yield and high optical purity. The economical benefit among others is the avoidance of preparation of unwanted isomer.

The route of synthesis of the various prior arts is briefly shown in Scheme-1.

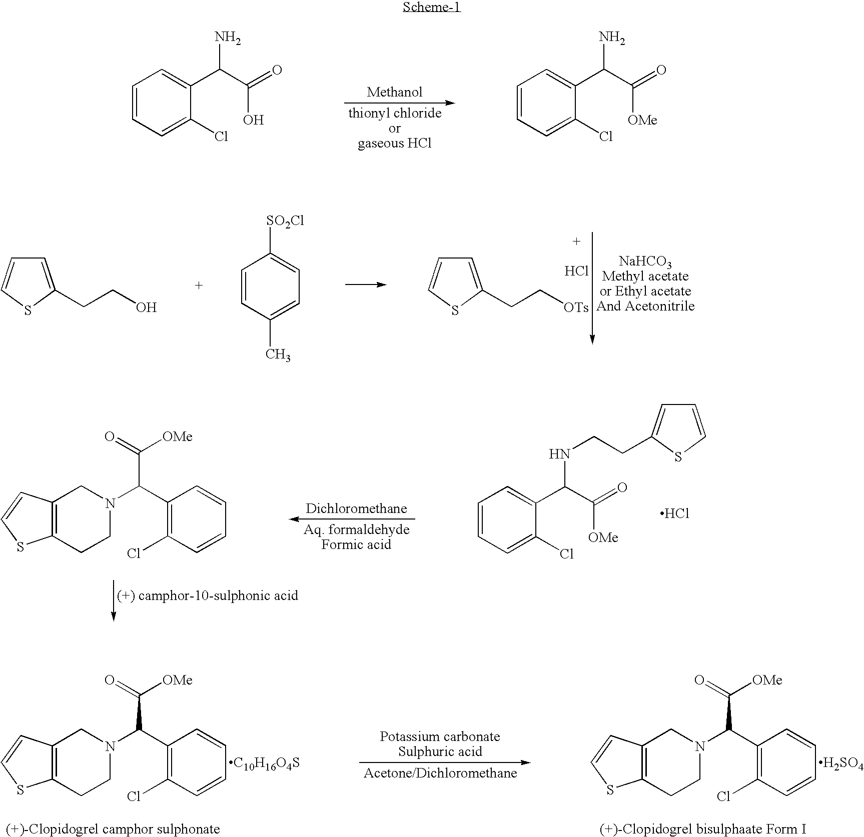

The following shortcomings are observed in the preparative methods as disclosed above for clopidogrel bisulfate Form-1.
(1) In all the above disclosed methods the isomer separation is done by using (+)-10-camphor sulphonic acid and moreover it is achieved in the final stage, which proves to be commercially costly.
(2) Poor isolated yields.
(3) Impurity profile in camphor sulfonate compound is difficult to control.

The main problem with the preparation of clopidogrel is the presence of a therapeutically inactive enantiomer, the (R) enantiomer. The presence of the (R) enantiomer results in contamination of the main product, and reduces the yield by being a waste product. Hence, there is a need in the art to prepare the (S) enantiomer of Clopidogrel substantially free of the (R) enantiomer in a facile manner suitable on an industrial scale.

### OBJECTIVES OF THE INVENTION

The objective of the invention is to ameliorate one or more problems associated with prior art.
The main objective of the present invention is to prepare clopidogrel bisulphate in economically viable manner.
Another objective of the invention is to provide polymorphic Form I in good isolated yields and with high enantiomeric purity.
Further objective of the present invention is to provide a process which is more convenient and industrially reproducible.

The above objectives and advantages will be clear from the description of the embodiment, which is outlined in a broad sense and alternatively featured in the present invention, so that those skilled in the art may better understand the detailed description of the invention that follows. Additional features of the invention will be described herein after, that form the subject of claims of the invention. Those skilled in the art should appreciate that they can readily use the disclosed concept and specific embodiment as a basis for carrying out the same process of the present invention and realize that such equivalent conception do not depart from the spirit and scope of the invention in its broadest sense.

### SUMMARY OF THE INVENTION

The present invention discloses an industrially viable and economically feasible process for preparation of clopidogrel bisulphate Polymorphic Form I.

In one aspect of the present invention, 2-chloro phenyl glycine was converted to 2-chloro phenyl glycine methyl ester using thionyl chloride in presence of methanol at a temperature of 50-65° C. This conversion was achieved using low molar volumes of thionyl chloride and methanol.

In another aspect, the optical isomer, (+) 2-chloro phenyl glycine methyl ester was separated from its (−) isomer using L-(+)-tartaric acid and the (+) tartrate salt thus obtained was converted into its free base using liquor ammonia. The (+) tartrate salt with high enantiomeric purity was achieved by repeatedly heating and cooling the reaction mass till the required enantiomeric purity obtained.

In further aspect, (+) 2-chloro phenyl glycine methyl ester free base was condensed with 2-thienyl para toluene sulphonate in presence of potassium bicarbonate in acetonitrile solvent at a temperature of 78-85° C. to obtain methyl (2S)-(2-chlorophenyl)[(2-thien-2-yl ethyl)amino]acetate to obtain the product in better quality and in good consistency. The product was isolated as its hydrochloride salt by treating with 30% solution of hydrochloric acid in ethyl acetate medium, which is further purified in a mixture of solvents toluene and methanol.

In yet another aspect, methyl (2S)-(2-chlorophenyl)[(2-thien-2-yl ethyl)amino]acetate hydrochloride was subjected to cyclization using para formaldehyde as a formylating agent, in aqueous medium with a very small quantity of HCl as a cyclization catalyst to obtain clopidogrel base, which was extracted into hexane and used as such for the preparation of Clopidogrel bisulphate Form I.

Furthermore, the present invention uses a novel method of precipitating the clopidogrel bisulphate Form I, directly from a mixture of solvents such as methanol and acetone in presence of sulfuric acid at a temperature ranging between 25 to 40° C., preferably at 30-32° C.

### DETAILED DESCRIPTION OF THE INVENTION

In accordance with the present invention, there is provided a process for preparation of clopidogrel bisulphate Form I. In the first stage, the conversion of 2-chloro phenyl glycine to 2-chloro phenyl glycine methyl ester hydrochloride using thionyl chloride in presence of methanol which is a more convenient operation on industrial level carry out stage I.

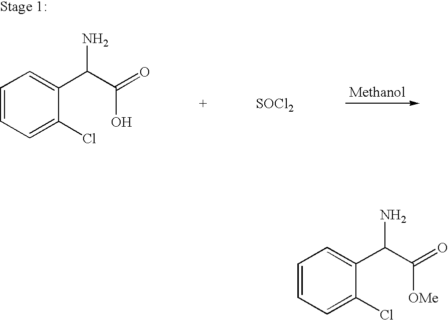

In the present invention, the thionyl chloride and methanol was used in lower molar ratios with reference to the starting compound 2-chloro phenyl glycine, whereby achieving the conversion to 2-chloro phenyl glycine methyl ester hydrochloride in greater yields with high purity. The reactant 2-chloro phenyl glycine, thionyl chloride and methanol were used in a molar ratio of 1:1:1. The reaction was carried out at a temperature ranging 50-65° C. for 5-6 hrs to ensure the total conversion. This ester was directly used for the second stage without carrying for any further purification.

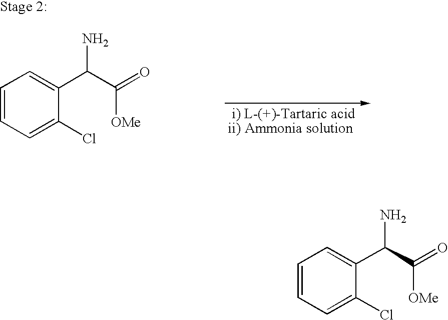

In the 2nd stage of synthesis, the optical isomer,(+) 2-chloro phenyl glycine methyl ester was separated from its (−) isomer using L-(+)-tartaric acid and the resolution was carried out at 50-55° C. and continued till the desired enantiomeric purity level achieved. This reaction was accomplished by repeated heating and cooling of the reaction mass and drawing the samples at designed intervals till proper quality parameters are achieved. The reaction was completed within a period of 45-50 hrs with desired entiomeric purity. The enantiomeric purity at this stage is highly important and the impurity profile as described in Pharmacoepia is to be matched, particularly with respect to the R-enantiomer. The (+)-tartrate salt thus obtained was used directly used for next stage, without subjecting the compound for further purification/crystallization techniques. Further the tartrate salt was converted into (+) 2-chloro phenyl glycine methyl ester free base using liquor ammonia.

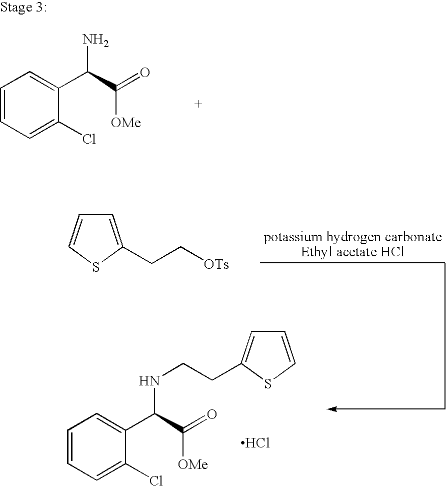

In the 3rd stage, the (+) 2-chloro phenyl glycine methyl ester free base was condensed with 2-thienyl para toluene sulphonate in presence of potassium bicarbonate in acetonitrile solvent at a temperature of 78-85° C. The reaction was maintained at the same temperature for 40 hrs to obtain methyl (2S)-(2-chlorophenyl)[(2-thien-2-yl ethyl)amino]acetate in better quality with consisting yields. The product was isolated as its hydrochloride salt by treating with 30% solution of hydrochloric acid in ethyl acetate medium. The crude product thus obtained was subjected to crystallization in a mixture of toluene-methanol in a ratio of 4:1 to achieve the product in high purity

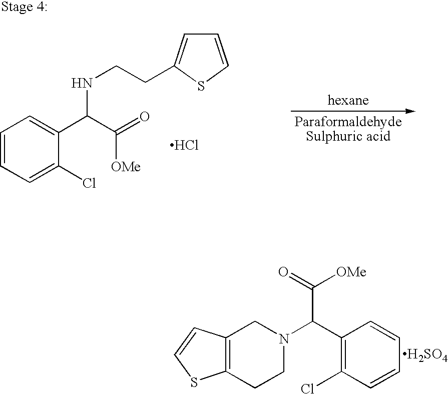

In the 4th stage, methyl (2S)-(2-chlorophenyl)[(2-thien-2-yl ethyl)amino]acetate hydrochloride was subjected to cyclization using para formaldehyde as formylating agent with a very small quantity of HCl as cyclization catalyst to obtain clopidogrel base. The cyclization reaction was carried out at 80-85° C. for 1-2 hrs till the starting material was absent on TLC. The clopidogrel base was extracted into hexane and used as such for the preparation of Clopidogrel bisulphate Form I.

Stage 5: The invention uses a novel method of precipitating the clopidogrel bisulphate Form I directly from a mixture of methanol and acetone in presence of sulfuric acid at a temperature ranging between 25 to 40° C., preferably at 30-32° C. The product clopidogrel bisulphate Form I, thus obtained was characterized and confirmed by the XRD, IR and DSC data. The impurity profile in the product obtained was well below the limits prescribed in the Pharmacopeia.

The present invention is further illustrated byway non-limiting examples, which does not limit the scope of the invention.

### EXAMPLE 1

2-chlorophenyl glycine methyl ester: Alpha amino(2-chlorophenyl)acetic acid (100 Kgs) is taken along with methanol (100 lit) and cooled to 0-5° C. Thionyl chloride (98 Kgs) was added to this in 2 hours at 0-5° C. The mass was heated slowly to 50-55° C. and maintained for 6 hours. The excess thionyl chloride along with the solvent are distilled off under reduced pressure. Water (450 lit) was added to the residue and stirred for 1 hour. The aqueous solution was washed with toluene to remove unwanted impurities. The pH of the aqueous layer was adjusted to 7.0-7.5 with liquor ammonia. The neutral aqueous layer was extracted with dichloro methane in 2 lots of 250 lit each. The organic layer was distilled to recover the solvent under educed pressure. 95 Kgs of 2-chlorophenyl glycine methyl ester was obtained as oily residue. The residue was used as such for the next step.

### EXAMPLE 2

(+) tartrate salt of 2-chlorophenyl glycine methyl ester: Methyl alpha amino(2-chlorophenyl)acetate (100 Kgs) was dissolved in acetone (72 lit). This solution was added to a suspension of L(+)-tartaric acid in methanol (400 lit) at 30-35° C. The reaction mass was stirred for 12 hours. The mass was cooled to 20-22° C. when solid was observed. The mass was again heated to 50-55° C.; kept for 2 hours and cooled again to 20-22° C. An aliquot sample was taken and tested for the melting range and enantiomeric purity. The process of heating and cooling was repeated for 8-10 times till the desired enantiomeric purity is achieved. (>99%). 87 Kgs of (+) tartrate salt of 2-chlorophenyl glycine methyl ester was obtained with enantiomeric purity of 99.0% and above.

[α]_{D}/20=+95.5° (c=1 in methanol)

M.P.=167 to 170° C.

### EXAMPLE 3

(+)-2-chlorophenyl glycine methyl ester: (+) Tartrate salt of 2-chlorophenyl glycine methyl ester (90 Kgs) was mixed with water (450 lit) and treated with ammonia solution (20%; 45 lit) till the pH of the reaction mass in the range of 7.0-7.2. Dichloromethane (270 lit) was added and stirred for 30 minutes. The organic layer was separated and the aqueous layer was extracted with dichloromethane (50 lit) twice. The organic layers are combined and distilled the solvent under reduced pressure to obtain the product, (+)-2-chlorophenyl glycine methyl ester (47 Kg) as oily residue.

### EXAMPLE 4

(+)-Methyl alpha-(2-thienyl ethyl amino)-(2-chlorophenyl)acetate hydrochloride: 50 Kg of (+)-2-chlorophenyl glycine methyl ester was dissolved in acetonitrile (250 lit). To this solution, potassium bicarbonate (75 Kg) and 2-thienyl ethyl para toluene sulphonate (100 Kg) was added at 25-30° C. The reaction mass was heated upto 78-80° C. and maintained for 40 hours. The solvent was distilled off under reduced pressure to yield a residue. To this residue, water (75 lit) and ethyl acetate (250 lit) were added and stirred for 1 hour. The organic layer was separated, added hydrochloric acid (30% solution; 25 lit) to the reaction mass till the pH of 1.2-1.5 attained. The reaction mass was centrifuged to obtain (+)-methyl alpha-(2-thienyl ethyl amino)-(2-chlorophenyl)acetate hydrochloride in a crude form. The product was dried and subjected to crystallization in a mixture of toluene-methanol (4:1) ratio to achieve 99.5% enantiomeric purity.

Yield: 65 Kg.

[α]_{D}/20=+110-112° (c=1 in methanol).

Enantiomeric purity: 99.5%

M.R=184-186° C.

### EXAMPLE 5

Clopidogrel bisulfate Form I: 50 Kgs of (+)-methyl alpha-(2-thienyl ethyl amino)-(2-chlorophenyl)acetate hydrochloride was taken in water (250 lit). Added para formaldehyde (17.7 Kgs) and a catalytic quantity of hydrochloric acid (0.5 lit). The reaction mass was heated to 80-85° C. and maintained for 1 hour till the starting material absent on TLC. The reaction mass was cooled to 25° C. and added n-hexane (250 lit). The pH of the reaction mass was adjusted to 2.4 to 2.7 and stirred for half an hour and the hexane layer was separated. The aqueous layer was extracted 3 times with n-hexane (100 lits each). The hexane layers are combined and distilled under reduced pressure to obtain oily residue. This oily residue was dissolved in a mixture of acetone and methanol (10:1) (165 lit), treated with activated carbon and filtered to remove unwanted color. The filtrate was cooled to 0-5° C. and pure sulphuric acid (8.95 Kgs) was added slowly in a period of 3 hours. The reaction mass was heated slowly to 25-30° C. and stirred for 12 hours. The precipitate formed was filtered and dried.

35 Kgs of Clopidogrel bisulphate Form I was obtained.

[α]_{D}/20=+55° (c=1 in methanol)

Enatiomeric purity: 99.6%

M.R=184-186° C.

The characterization of compound was performed by XRD and IR and the values obtained are in accordance with those reported in the literature.

What is claimed is:

1. The method of preparation of clopidogrel bisulphate Form I characterized in that the process;
a. using low ratio of 1:1 of 2-chloro phenyl glycine to thionyl chloride to get 2-chloro phenyl glycine methyl ester in presence of methanol; and
b. directly precipitating clopedogrel bisulfate form 1 from mixture of solvents such as methanol and acetone in presence of sulfuric acid at a temperature ranging between 25° to 40° C., more preferably 30°-32° C.

2. The method of preparation of clopidogrel bisulphate Form I according to claim 1, wherein the process steps comprises of;
c. reacting 2-chlorophenyl glycine with thionyl chloride in methanol to obtain 2-chlorophenyl glycine methyl ester, wherein the ratio of 2-chlorophenyl glycine to thionyl chloride and methanol are 1:1:1;
d. resolving the 2-chlorophenyl glycine methyl ester into its optical isomers using L(+) tartaric acid in a solvent mix of acetone and methanol, to obtain the (+) tartrate salt of 2-chlorophenyl glycine methyl ester with high enantiomeric purity;
e. generating the free base (+)-2-chlorophenyl glycine methyl ester from its tartrate salt by neutralizing the salt using liquor ammonia;
f. condensing the (+)-2-chlorophenyl glycine methyl ester with 2-thienyl ethyl para toluene sulphonate in presence of potassium bicarbonate at a temperature of 25°-30° C., and isolating the product as hydrochloride salt in a conventional manner;
g. reacting the (+)-methyl alpha-2-(thienyl ethyl amino)-(2-chlorophenyl)acetate hydrochloride with para formaldehyde in aqueous medium using catalytic amount of mineral acid as a cyclizing agent to obtain clopidogrel base; and
h. precipitating the clopidogrel bisulphate Form I, directly from a mixture of acetone and methanol by treating with sulphuric acid.

3. The method of preparation of clopidogrel bisulphate Form I according to claim 2 (b), wherein said reaction is carried out by repeated process of heating the reaction mass upto 50°-55° C. and cooling to 20°-22° C. till to achieve the required enantiomeric purity.

4. The method of preparation of clopidogrel bisulphate Form I according to claim 2 (d), wherein said product is further crystallized from a mixture solvents toluene and methanol.

5. The method of preparation of clopidogrel bisulphate Form I according to claim 4, wherein said solvents are used in a ratio of 4:1.

6. The method of preparation of clopidogrel bisulphate Form I according to claim 2 (e), wherein said mineral acid is hydrochloric acid.

7. The method of preparation of clopidogrel bisulphate Form I according to claim 2 (e), wherein the reaction is carried out at a temperature ranging at 80-85° C.

8. The method of preparation of clopidogrel bisulphate Form I according to claim 2 (f), wherein said reaction is carried out at a temperature of 25°-40° C.

9. The method of preparation of clopidogrel bisulphate Form I according to claim 2 (f), wherein the mixture of solvents used is acetone and methanol in a ratio of 10:1.
